(19) United States
(12) Patent Application Publication
(10) Pub. No.: US 2008/0269698 A1
(43) Pub. Date: Oct. 30, 2008

(54) OSTOMY APPLIANCE COLLECTOR

(75) Inventors: Conor Alexander, Norfolk, VA (US); Bhavani Veerapaneni, Princeton, NJ (US); Kenneth Johnsen, Piscataway, NJ (US)

Correspondence Address: CONVATEC INC., 100 HEADQUARTERS PARK DRIVE, SKILLMAN, NJ, 08558, US

(73) Assignee: Bristol-Myers Squibb Company, New York, NY (US)

(21) Appl. No.: 11/739,737

(22) Filed: Apr. 25, 2007

(51) Int. Cl. A61F 5/445

(52) U.S. Cl. 604/332; 264/101

(57) ABSTRACT

An ostomy appliance comprising a collection device for collecting body waste. At least a portion of the collection device is thermoformed from a sheet of plastics material, e.g., vacuum formed or pressure formed. The collection device may be configurable between a compact, stowed condition and an expanded deployed condition. The thermoformed portion is thermoformed in the deployed condition. The collection device has a depth:diamater ratio of at least 1:1. The ostomy appliance is a controlled evacuation appliance, or a collection pouch.

### FIELD OF THE INVENTION

The present invention relates to a collector device for collecting body waste in an ostomy appliance. The collector device is of a type that is expandable from a compact form, in which the collector device is stowed initially, to an expanded form, in which the collector device is deployed for collecting the body waste. The collector device is especially suitable for inclusion in a controlled evacuation ostomy appliance, but the invention is not limited exclusively to this. The invention may be directed to a collection device, and/or to an ostomy appliance incorporating a collection device. The term “ostomy” includes one or more of colostomy, ileostomy and urostomy.

### BACKGROUND TO THE INVENTION

U.S. Pat. Nos. 6,723,079 and 6,689,111, U.S. Published Patent Application 2004181197, Published PCT Application WO-A-02/058604, and DE-A-19921555 describe various designs of ostomy appliance that include expandable ostomy collection devices. Each collection device is tubular, and is provided initially in an axially collapsed form in which the collection device is stowed in the appliance. The collection device is expandable from its initial form, into an expanded form for collecting and containing body waste discharged from the stoma.

The first three of the above specifications describe ostomy appliances in the form of controlled evacuation devices that allow the user to control when a stomal discharge will take place. The appliances block or occlude the stoma, such that body waste is stored temporarily in the portion of the bowel leading to the stoma. When the blocking or occluding portion of the appliance is removed and the collection device expanded, body waste is discharged into the collection device. Controlled evacuation devices offer the potential for an ostomate to regain some control over bodily function, by controlling when a discharge of body waste take place.

One of the desired features of all of the above appliances is that the appliance should preferably be compact, yet be able to provide a significant collection capacity. In one non-limiting form, the present invention seeks to provide a form of collection device that is especially suitable for such appliances by enabling the collection device initially to have a highly compact form without interfering with other functional parts of the appliances, yet also provide a substantial collection volume for body waste, e.g., when the collection device is deployed to its expanded condition.

### SUMMARY OF THE INVENTION

Broadly speaking, one aspect of the invention provides a collection device for an ostomy appliance, at least a portion of the collection device being thermoformed.

The thermoformed portion is thermoformed from plastics sheet or film.

The terms “thermoformed” or “thermoforming” is used herein to refer to any process in which a pressure differential between opposite sides of the plastics sheet, is used to draw or force the sheet against a shape defining Form. In one aspect of the invention, the thermoforming is specifically vacuum forming or pressure forming.

In one non-limiting form, the invention can enable a collection device to be provided that (i) is formed from plastics sheet, and (ii) has a depth to transverse dimension ratio, or so-called “aspect ratio”, (e.g., depth to diameter ratio) of at least 1:1. The ratio may be far greater than 1:1.

The process of thermoforming the collection device (portion) is carried out using a male or female Form. The term “Form” is used herein to refer to the shape-defining portion of the apparatus for thermoforming an article to a desired shape defined by the Form.

The collection device may be collapsed or collapsible into a compact form. For example, if the collection device has a tubular bag form, the collection device may be collapsed into a generally flat, or low profile, annulus.

The collection device may be expanded or distended from the compact form to a deployed form. The deployed form may be substantially the same shape in which the collection device is originally thermoformed. The use of thermoforming can enable the expanded shape to be well-defined and/or controlled, in contrast to, for example, a flexible collection device with little or no control over its shape as it fills.

The invention provides a thin-walled tubular bag, that may be collapsed axially, to form a compactly folded cylinder. The film used to form the bag may be chosen for its flexibility, ability to be thermoformed into a flexible shape, ability to be welded to other components, and/or odor barrier properties.

The collection device is especially suitable for use in a controlled evacuation device, but it is not limited exclusively to this. Additionally, or alternatively, the collection device is especially suitable for use in an ostomy appliance using a deployable collection device, but the invention is not limited exclusively to this.

While it is believed that principal features of the invention have been defined above and/or in the claims, further or alternative features of the invention may be disclosed in the following description and/or drawings. The Applicant claims protection for any novel feature or idea described herein and/or illustrated in the drawings, whether or not emphasis has been placed thereon.

### BRIEF DESCRIPTION OF THE DRAWINGS

Non-limiting embodiments of the invention are now described, by way of example only, with reference to the accompanying drawings.

FIGS. 1A and 1B are schematic sections showing a controlled evacuation ostomy appliance employing a deployable collection device, in its blocking and non-blocking conditions, respectively.

FIGS. 2A, 2B and 2C are schematic views showing a first embodiment illustrating the principle of vacuum forming the collection device from plastics sheet.

FIGS. 2D, 2E and 2F are schematic views illustrating other examples of Forms.

FIG. 3 is a schematic section illustrating a second embodiment of vacuum forming.

FIG. 4 is a schematic section illustrating a third embodiment of vacuum forming.

FIGS. 5A and 5B are schematic sections showing a first example of the collapsible Form in its compact and deployed conditions, respectively.

FIG. 5C is a schematic perspective view of a collection device formed by the Form.

FIGS. 6A and 6B are schematic sections showing a second example of collapsible Form in its compact and deployed conditions, respectively;

FIG. 7A is a schematic perspective view showing a coupling ring pre-attached to a sheet before vacuum forming.

FIG. 7B is a schematic section showing the Form during vacuum forming.

FIG. 8A is a schematic section showing integral attachment of a plastics member to a sheet during vacuum forming.

FIGS. 8B-E are schematic perspective views showing the integral attachment process.

FIGS. 9A and 9B are schematic sections illustrating vacuum forming using a female Form.

FIGS. 10A and 10B are schematic side and front perspective views of a first example of pouch including a front vacuum formed portion.

FIG. 11 is a schematic side view of a second example of pouch including front and rear vacuum formed portions.

### DETAILED DESCRIPTION OF PREFERRED EMBODIMENTS

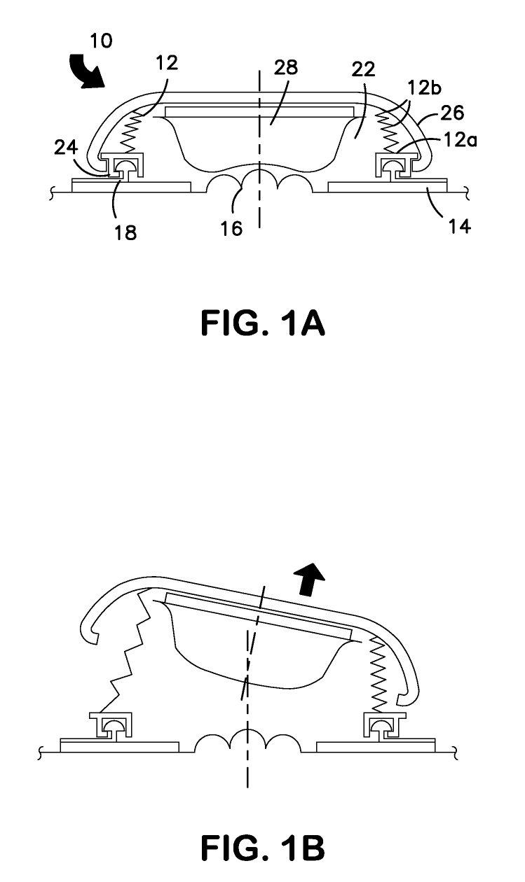

Referring to FIG. 1, an ostomy appliance 10 may be illustrated employing a deployable collection device 12 for body waste. In the present embodiment, the appliance 10 is in the form of a controlled evacuation device for controlling discharge of body waste from the stoma, but the collection device 12 may equally be used in other ostomy appliances without controlled discharge. Also, in the present embodiment, the collection device 12 is of a type that is deployable from a compact condition, to a deployed condition, but the collection device 12 need not be of such a “deployable” type in all embodiments.

The appliance 10 generally comprises:
(a) an adhesive faceplate 14 for attachment to the wearer's skin around a stoma 16, and carrying a first coupling ring 18;
(b) the collection device 12 having a proximal end 12a and a distal end 12b. The collection device may comprise at least one aperture 20, for example, at the proximal end. The collection device 12 may optionally comprise a second aperture 22, for example, at the distal end;
(c) an intermediate coupling ring 24 mateable with the first coupling ring 18, and attached to the proximal end 12a of the deployable collection device 12;
(d) a cover 26 mateable with, or removeably attached to, the intermediate coupling ring 24, and attached to the distal end 12b of the deployable collection device 12; and
(e) a stoma seal 28 for creating a seal in or against the stoma 16 for obstructing the discharge of at least solid body waste from the stoma 16.
   The seal 28 may be carried directly or indirectly by the cover 26.

The appliance has two operating conditions. The first, illustrated in FIG. 1A, is the blocking (or non-discharge) condition, in which the cover 26 is mated to or attached to the intermediate coupling ring 24, such that the collection device 12 is held in a stowed condition between the cover 26 and the intermediate coupling ring 24, and the seal 28 is positioned such that it may obstruct discharge from the stoma 16. The blocking condition is the condition in which the appliance 10 is initially supplied to, and worn by, a user. The second condition, illustrated in FIG. 1B, is the non-blocking (or discharge) condition, which the user selects when the user desires to discharge waste from the stomal. In the second condition, the cover 26 is separated from, and displaced away from, the intermediate coupling ring 24 in order to remove the seal 28 from the stoma, and to distend the collection device 12 to a deployed condition. In the deployed condition, the collection device 12 provides a substantial collection volume for collecting and containing stomal discharge.

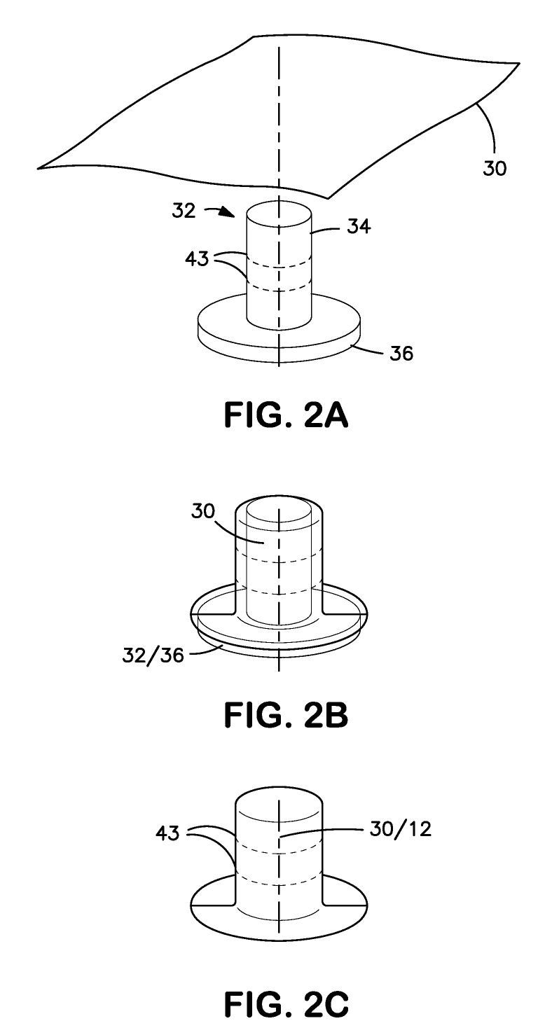

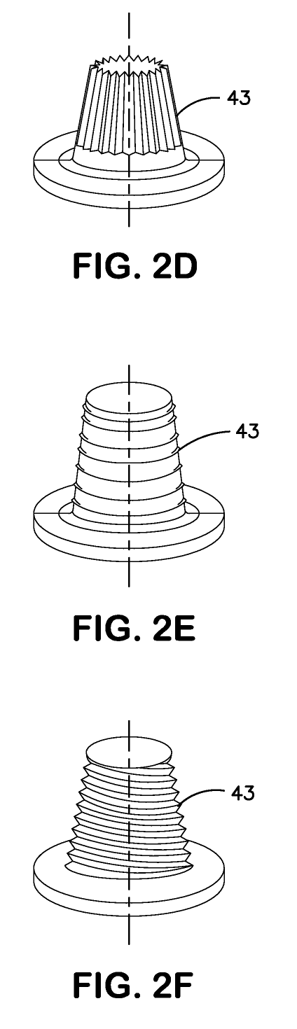

Referring to FIGS. 2A, 2B, 2C and 3, the collection device 12 generally comprises a thermoformed bag, made of flexible plastics material. Hereafter, the terms “collection device” and “bag” may be used interchangeably (although the term bag is used generally to describe the article during manufacture, and the term collection device is used to describe the article after manufacture). The especially preferred thermoforming techniques for which the invention is especially suitable is vacuum forming and/or pressure forming. However, other thermoforming techniques may be used that generate a pressure differential on either side of a sheet in order to draw the sheet against a shape defining Form. The following examples describe vacuum forming, but the same principles may be used in other thermoforming techniques.

The bag is vacuum formed in its deployed shape. Thereafter, the bag is collapsed into its stowed shape for use in the appliance 10. The bag has a generally hollow tubular shape, although other shapes may be used as desired. The use of the thermoforming process described herein enables the collection device 12 (in its deployed shape) to have a depth-to-diameter ratio greater than 1:1, for example, at least 2:1, or at least 3:1, or at least 4:1, or at least 5:1, or at least 6:1, or at least 7:1, or at least 8:1, or at least 9:1, or at least 10:1.

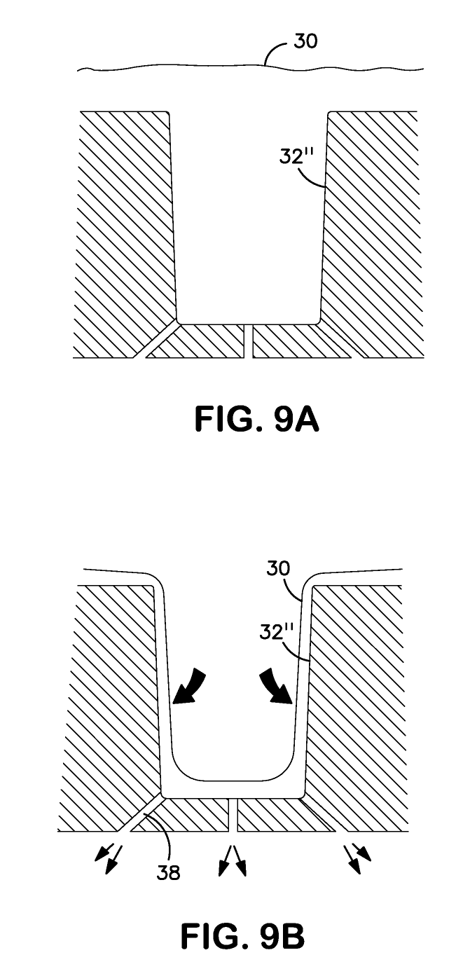

The bag is vacuum formed from a sheet (or film) 30 of flexible plastics material, vacuum drawn against a Form 32. In FIG. 2, the Form 32 may be a male Form, although a female Form may be used as desired (and described later with respect to FIG. 9). The Form 32 comprises an upstand 34 projecting from a base 36. One or more suction ports 38 are formed in the upstand 34 and/or the base 36, for applying a vacuum in order to draw the plastics sheet 30 against the Form 32.

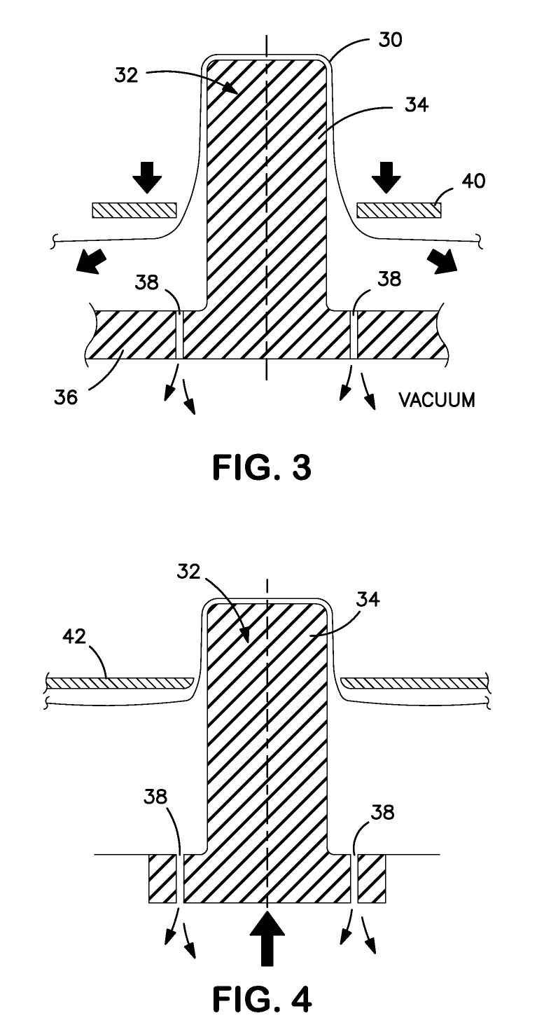

One issue that may be encountered when vacuum forming the collection device 12 with a high aspect ratio shape, may be the formation of pleats and wrinkle imperfections in the bag. These imperfections result from the formation of pleats and wrinkles in the sheet 30 as the sheet 30 is drawn towards the bottom of the Form 32. Such imperfections are undesirable aesthetically and/or technically, for example, if the imperfections interfere with subsequent processing of the bag after vacuum forming. The formation of pleats and/or wrinkles may optionally be controlled or substantially eliminated, by using a female structure, such as a washer structure 40 (FIG. 3). The washer structure 40 has an inner diameter that is slightly larger than the outer diameter of the upstand 34. The washer structure 40 is placed over the sheet 30 on the Form 32 prior to applying the vacuum to the Form. The washer structure 40 causes the sheet 30 to stretch and drape more evenly around the Form. Alternatively, referring to FIG. 4, the washer structure 40 may be replaced by an apertured plate 42. The apertured plate 42 remains generally stationary, and the sheet 30 is placed generally flat against the aperture plate 42. The upstand 34 of the Form 32 is advanced against the sheet 30, at a position generally in register with the aperture in the plate 42, to displace the film through the aperture and around the Form 32. The diameter of the aperture is slightly larger than the diameter of the upstand 34. The plate 42 guides and stabilizes the sheet 30 in a similar manner to the washer structure 40 described above.

The upstand 34 has a generally smooth exterior surface. The vacuum formed bag has generally smooth shaped walls (subject to any wrinkling and pleating as explained above). When the collection device 12 is collapsed to its compact form, the side wall of the bag collapses generally randomly, along random fold lines.

Alternatively, as indicated in phantom at 43 in FIGS. 2A-C, the exterior surface includes one or more surface features for imparting a corresponding shape feature to the vacuum formed bag. Such a shape feature stabilizes the bag during collapsing, such that the bag can collapse in a predetermined way and/or with folds at predetermined positions and/or along predetermined fold lines. The shape features include axial features and/or circumferential features and/or spiral features. The shape features appear to have a random pattern, or a degree of uniformity or non-randomness. By way, of example, the shape features include one or more circumferential ribs (projecting or recessed), or folds. The shape features are small, such that they do not interfere with removal of the Form 32 from the vacuum formed bag. Alternatively, if larger shape features are desired, then an expanding Form 32′ may be suitable (as described later). FIGS. 2D-F illustrates example Forms for creating such surface shape features.

The upstand 34 of the Form 32 is of generally parallel cylindrical shape. The bag formed using such a Form 32 is of about equal diameter along its entire length, such that the ends are of about the same diameter as each other (subject to possible inward rounding at the end of the bag corresponding to the tip of the upstand 34, and possible outward rounding at the end of the bag corresponding to the point at which the upstand 34 meets the base 36).

Alternatively, the upstand 34 has a narrowing shape, such that the upstand 34 narrows in a direction from the base 36 to the tip of the upstand 34. The upstand 34 narrows progressively and/or with one or more steps. Such a shape of Form 32 produces a bag having one end (e.g., the distal end 12b) adjacent to the tip of the upstand 34 narrower in diameter than the opposite end (e.g., the proximal end 12a) adjacent to the base 36. Such a narrowing shape is beneficial in aiding removal of the bag from the Form 32 after vacuum forming.

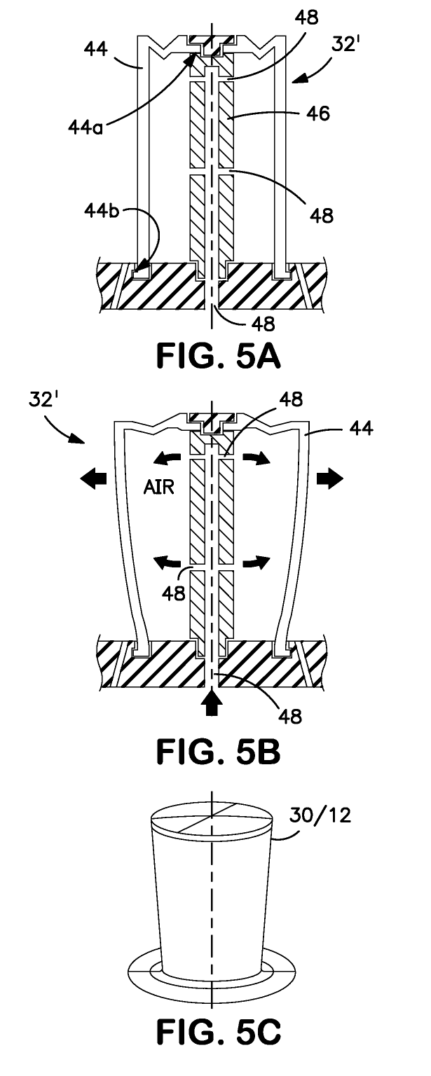

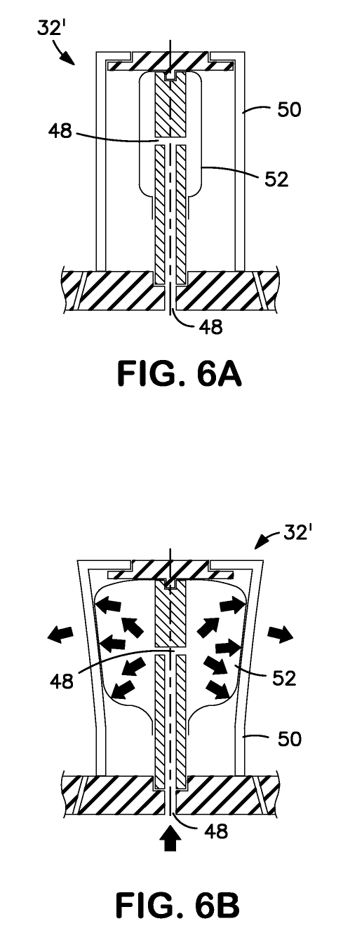

In other cases, it is desirable to create a bag having shape in which the diameter at a first arbitrary point at a first distance from the base 36 is larger than the diameter at a second arbitrary point at a second distance from the base 36 larger than the first distance. Such bag is formed by a Form 32 of undercut shape. It is difficult to form such a bag shape using a fixed-shape Form 32 without causing undesirable expansion of the bag when the bag is removed from the Form 32 (or vice versa). In such a case, an expanding Form 32′ is appropriate, as illustrated in FIGS. 5 and 6. The term “expanding” Form is used herein to refer to any Form 32′ that is capable of changing shape between a deployed shape (e.g., the shape of the Form for the vacuum forming process), and a compacted shape (e.g., a smaller shape to allow easy removal of the Form 32′ from within the vacuum formed bag). The expanding Form 32′ has a natural shape with respect to which the expanding Form 32′ is capable of resilient deformation. For example, the natural shape could be either of the deployed and compacted shapes, or an intermediate shape.

Referring to FIG. 5, a first example of expanding Form 32′ comprises an integral, elastic, inflatable Form member 44 that is sealed at both ends 44a and 44b. The Form member 44 is supported by a central column 46 projecting from the base 36. The natural shape of the Form member 44 (FIG. 5a) corresponds to its contracted shape. The Form member 44 is pressurized via inflation ports 48, to expand the Form member 44 to its deployed shape (FIG. 5b). The deployed shape is an inverted, smoothly tapered frusto-cone, that narrows in diameter from its tip to the base 36. After the bag has been vacuum formed around the Form member 44 in the deployed shape of the Form member 44 (FIG. 5c), the Form member 44 is depressurized to allow the Form member to return to its contracted shape, to allow the vacuum formed bag to be easily removed from the Form 32′, without substantially expanding or distorting the shape of the vacuum formed bag.

Referring to FIG. 6, a second example of expanding Form 32′ comprises an external Form skin 50, and an internal expansion bladder 52. In a similar manner to the first example of FIG. 5, the natural shape of the Form skin 50 is its contracted shape. By applying a pressure via the inflation ports 48, the internal inflation bladder 52 is pressurized to deform the Form skin 50 outwardly to its deployed shape. When the inflation pressure is removed, the Form skin 50 is allowed to return elastically to its contracted shape.

Suitable materials for the Form 32′ include elastomers that are typically used in industrial and medical product applications. These include vulcanized rubbers such as BUNA and Viton, silicone rubbers, and certain thermoplastic elastomers. It is also possible to create an expanding/contracting Form 32′ using a flexible, non-elastic material. By virtue of the shape and material properties, such a Form 32′ may be expanded and relaxed at appropriate times. In the relaxed state, the material should be sufficiently flexible to allow easy removal of the vacuum formed bag. Thin, flexible, heat resistant materials, such as mylar may be suitable for such a Form 32′.

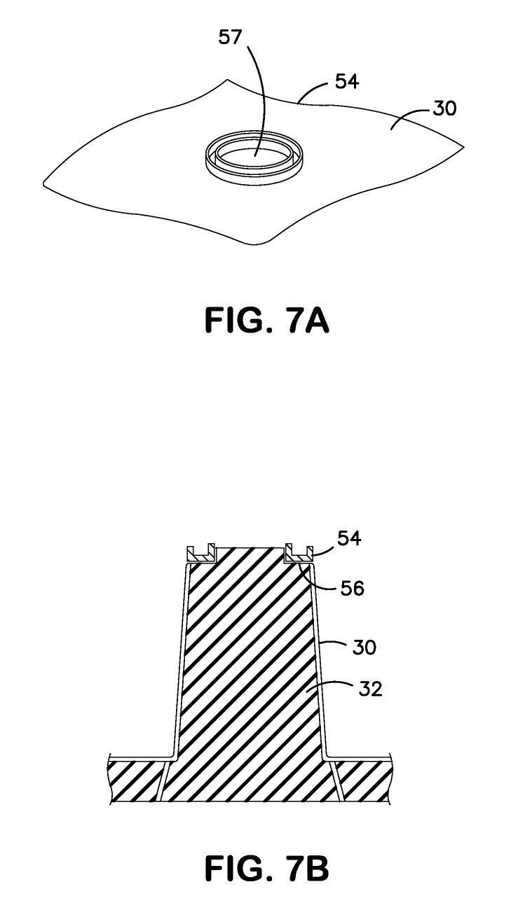

In general, other components or accessories may be attached to the bag, either as part of the vacuum forming process, or as an additional step before or after vacuum formation. For example, referring to FIG. 7(a), a thermoplastic coupling ring 54 (which may be the intermediate coupling ring 24) is attached to the sheet 30 prior to vacuum forming. The Form 32 may include a recess 56 (FIG. 7b) for at least partly accommodating the coupling ring 54. The coupling ring serves to positively locate the sheet 30 with respect to the Form 32. The portion 57 of the sheet material 30 inside the coupling ring 54 is removed either before or after the vacuum forming process. If the portion 56 of material is removed before vacuum forming, the coupling ring 54 provides a seal between the sheet 30 and the Form 32, so that a vacuum may still be applied to the remainder of the sheet 30.

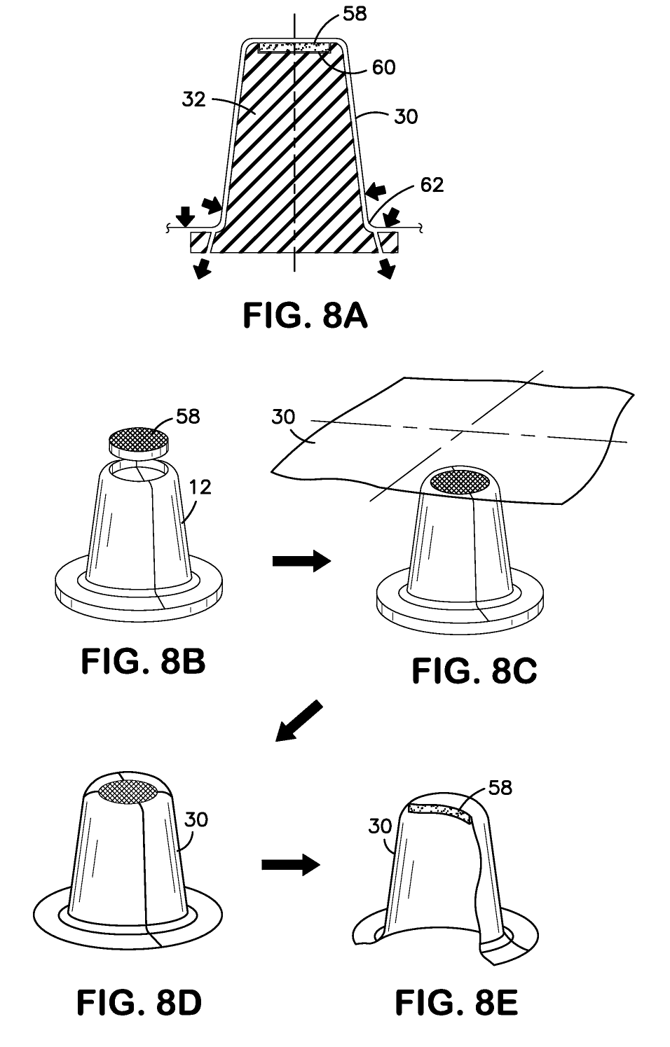

FIGS. 8A-E illustrates integral attachment of a thermoplastic component 58 during the vacuum forming process. The thermoplastic component 58 is positioned in, against or around, the Form 32 before the sheet 30 is draped over the Form. The Form 32 includes a recess 60 for at least partly accommodating the component 58. During the vacuum forming process, the sheet 30 is forced into contact with the component 58, and bonds thereto by virtue of heat and the pressure generated by the vacuum forming process. Although, the component 58 may be illustrated in FIG. 8 as being positioned at the distal end of the bag, this is merely by way of example. The component 58 may be positioned at any desired location, for example, at the open end of the bag. Such a location may be suitable for integrally attaching the intermediate coupling ring 24 of the appliance 10.

Alternatively, a component may be attached to the bag after the vacuum forming process. For example, a mouth portion 62 (FIG. 8A) of the bag corresponding to the junction between the upstand 34 and the base 36 typically includes a peripheral flange or other outward facing planar surface. Such a flange may be suitable as a planar surface for attaching to another component (e.g., the intermediate coupling ring 24) or to another sheet.

A preferred method of attaching the collection device 12 to another component (such as the intermediate coupling ring 24 or the cover 26) is by welding. Such welding includes integral welding (as part of the vacuum formation process), heat welding, radio frequency welding, or laser welding. However, other attachment techniques, such as adhesive bonding, may be used as desired.

The sheet 30 may made of any suitable material, or combination (e.g., laminate) of materials that can be vacuum formed as desired in the present invention. The sheet 30 may have odor barrier properties for obstructing transpiration of malodours through the plastics material. Additionally, or alternatively, the material may be selected so as to have desired weldable properties, for example, so as to be weldable to other components of the appliance 10.

Suitable plastics materials (with good welding and odor barrier properties) may, by way of example only, include low density polyethylene (LDPE), ethylene vinyl acetate (EVA), and multi-layer films, such as ethylene-vinyl-acetate/poly-vinylidene-chloride/ethylene-vinyl-acetate (EVA/PVDC/EVA). Ethylene vinyl alcohol may be substituted for PVDC if desired. Many other single or multiple layer films may be used as desired, such as polypropylene and/or nylon. The material may be at least partly biodegradable.

The sheet or film 30 has a thickness of at least about 0.01 mm. Additionally or alternatively, the thickness may be less than or equal to about 5 mm, or less than or equal to about 2 mm.

After vacuum forming, the bag is trimmed of excess sheet material, to leave only the material defining the desired bag shape of the collection device 12. The collection device 12 is collapsed from its originally formed shape to its compact shape, ready for storage or for assembly into the appliance. The collection device 12 is collapsed around a mandrel or other former inserted into the collection device 12. Use of the mandrel ensures that the cylindrical side wall of the collection device 12 does not collapse into the central space that is to be occupied by the seal 28.

Although the embodiments of Forms 32 and 32′ described above have been illustrated as a male Form, the same principles may be applied using a female Form 32″, for example as illustrated in FIG. 9. The female Form 32″ may implement any of the male Form features described above. A female Form 32″ is especially suitable for forming bag shapes that may be difficult or even impossible to create using a male Form. The choice of male of female Form can be made according to the properties of the desired collecting device 12, and the desired manufacturing criteria.

Although the collection device 12 has been illustrated in the foregoing examples to have a circular shape, the above embodiments may use any closed-loop shape as desired, for example, a rectilinear shape. The examples hereafter illustrate oval or egg-shaped forms.

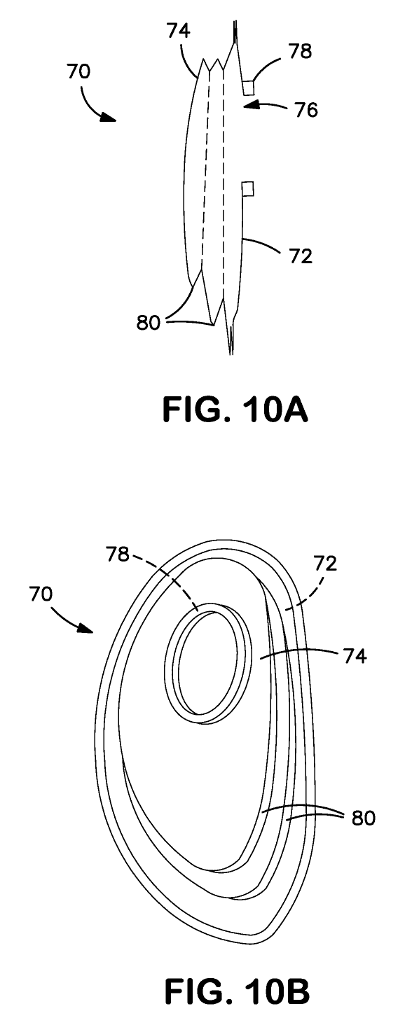

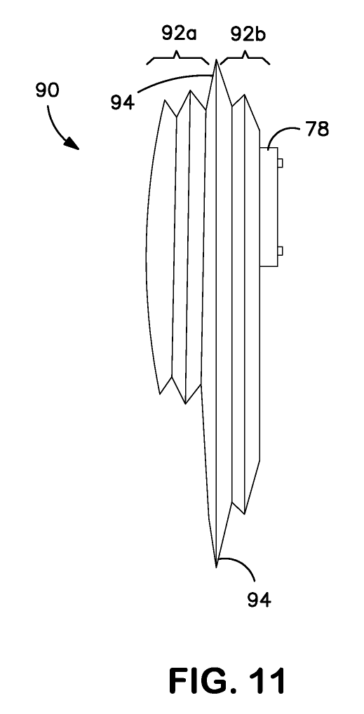

Although the principles of the invention have been illustrated in connection with a deployable collection device 12 for a controlled evacuation appliance 10, the invention is by no means limited to such an appliance. FIGS. 10 and 11 illustrate ostomy pouches 70 and 90 having collection devices incorporating vacuum formed portions.

Referring to FIG. 10, the ostomy pouch 70 comprises a generally flat, conventional rear wall 72, and a thermoformed portion 74 forming the front wall of the pouch 70. The rear wall 72 includes a stomal aperture 76 and a conventional attachment device 78 for attaching the pouch 70 to a wearer's body. The thermoformed portion 74 comprises concertina segments 80.

Referring to FIG. 11, thermoformed portions 92a and 92b are used to implement both the front and rear “walls” of the pouch 90. In this embodiment, the entire collection chamber (collection device) of the pouch 90 is thermoformed, and the concept of front and rear “walls” is referred to here merely to relate the pouch 90 to a conventional ostomy pouch comprising generally flat, flexible, front and back walls. The thermoformed portions 92a and 92b are vacuum formed as separate front and rear shells which are attached together along a mutual seam 94 after thermoforming, as it might be awkward to create the bag shape of FIG. 11 using a single Form.

A pouch 70 or 90 including at least a thermoformed portion 74, 92a or 92b, offers one or more of the following advantages:
(a) the thermoformed portion 74 or 92 is collapsed into a compact shape, so that the pouch 74 or 92 initially is not substantially greater than a conventional ostomy pouch;
(b) the thermoformed portion 74 or 92 expands to provide a pouch of substantial collection capacity;
(c) the thermoformed portion 74 or 92 provides an expandable portion that expands more efficiently and effectively than a conventional pouch wall;
(d) the thermoformed portion 74 or 92 provides a region of controlled shape expansion, instead of uncontrolled deformation of a conventional pouch wall; and/or
(e) the thermoformed portion 74 or 92 us shaped to define a specific contour, such as one or more progressively smaller shapes, or one or more progressively larger shapes.

The shape of the pouch 70 or 90 can be controlled to provide one or more regions of controlled expansion, by welding specific areas of facing pouch film and/or specific areas of the vacuum formed material, to one another, or by attaching these facing areas to each other by means of low-tack adhesive to create designated zones of controlled expansion.

It will be appreciated that the use of a thermoformed (e.g., vacuum formed, or pressure-formed) collection device (portion) as in the present invention enables an expandable collection device (portion) to be manufactured from sheet material, that has a desirably small compact shape, yet also provides a substantial collection volume when desired.

The foregoing description is merely illustrative of preferred forms of the invention. Many modifications, alternatives and equivalents may be used within the scope and/or spirit of the invention.

We claim:

1. An ostomy appliance comprising a collection device, at least a first portion of the collection device being thermoformed from a sheet of plastics material.

2. The ostomy appliance according to claim 1, wherein the thermoformed portion is a vacuum formed portion or a pressure formed portion.

3. The ostomy appliance according to claim 1, wherein said collection device is substantially entirely thermoformed.

4. The ostomy appliance according to claim 1, wherein said collection device is deployable, in use, from a compact shape to a deployed shape.

5. The ostomy appliance according to claim 4, wherein said first portion is thermoformed in a shape corresponding to said deployed shape.

6. The ostomy appliance according to claim 1, wherein the collection device has a generally tubular shape.

7. The ostomy appliance according to claim 1, wherein the collection device has ratio of depth to transverse-dimension of at least 1.

8. The ostomy appliance according to claim 1, wherein the collection device comprises at least one aperture.

9. The ostomy appliance according to 1, wherein the collection device comprises material selected as one or more of: polyethylene; polypropylene; nylon; polyvinyl chloride; ethylene vinyl acetate; poly vinyl alcohol (PVOH); polyvinylidene chloride (PVDC).

10. The ostomy appliance according to claim 1, wherein the ostomy appliance is a controlled evacuation appliance.

11. The ostomy appliance according to claim 1, wherein the ostomy appliance is a collection pouch.

12. The ostomy appliance according to claim 1, further comprising a second portion attached to the first portion.

13. The ostomy appliance according to claim 12, wherein the second portion comprises one or more of: a second collection device portion; a molded component; a coupling member; a flexible sheet.

14. The ostomy appliance according to claim 12, wherein the second collection device portion is a thermoformed portion.

15. The ostomy appliance according to claim 12, wherein the second portion is attached to the collection device by one or more of: integral attachment during the thermoforming process; a heat weld; an ultrasonic weld; a radio-frequency weld; a laser weld; an adhesive bond.

16. The ostomy appliance according to claim 1, wherein said collection device comprises surface features selected from one or more of: circumferential features, axial features; spiral features; random features.

17. The ostomy appliance according to claim 16, wherein said features are selected from one more of: ribs; folds; recurrent surface features.

18. The ostomy appliance according to claim 1, wherein the collection device comprises said first portion attached to a flexible sheet panel.

19. The ostomy appliance according to claim 1, wherein the collection device comprises said first portion attached to a second thermoformed portion.

20. A collection device for an ostomy appliance, at least a first portion of the collection device being a portion that is thermoformed from a sheet of plastics material.

21. The collection device according to claim 20, wherein the collection device is configurable between a compact condition and a deployed condition.

22. The collection device according to claim 21, wherein the first portion of the collection device is thermoformed in a shape corresponding to said deployed condition.

23. Apparatus for thermoforming at least a first portion of a collection device for an ostomy appliance, the apparatus comprising a shape-defining Form for thermoforming said first portion of the collection device from a sheet of plastics material.

24. The apparatus according to claim 23, wherein the Form is selected from: a male Form; and a female Form.

25. The apparatus according to claim 23, wherein the Form is selected from: a fixed-shape Form; and a collapsing/expanding Form.

26. The apparatus according to claim 23, wherein the Form is a male form, and the apparatus further comprises a female structure having an aperture dimensioned to receive the male Form with only a narrow gap therebetween.

27. A method of producing at least a first portion of a collection device for an ostomy appliance, the method comprising thermoforming said first portion from a sheet of plastics material.

28. The method according to claim 27, wherein the step of thermoforming comprises vacuum forming or pressure forming said first portion.

29. The method according to claim 27, wherein the collection device is configurable between a compact condition and a deployed condition, and wherein the step of thermoforming comprises thermoforming said first portion in a shape corresponding to the deployed condition.

30. The method according to claim 27, further comprising integrally attaching a second portion to the first portion during the step of thermoforming the first portion.

31. The method according to claim 30, wherein the second portion is selected as one or more of: a second collection device portion; a molded component; a coupling member; a flexible sheet.

32. The method according to claim 27, wherein the thermoforming is performed using a male Form and a female structure, the female structure comprising an aperture dimensioned to receive the male Form with only narrow gap therebetween, and wherein the method comprises moving at least one of the male Form and the female structure relative to the other, whereby the female structure smoothes the sheet around the male Form.
